(19) United States
(12) Patent Application Publication
(10) Pub. No.: US 2002/0168323 A1
(43) Pub. Date: Nov. 14, 2002

(54) Optimization of the molecular properties and formulation of proteins delivered by inhalation

(76) Inventor: Igor Gonda, Melbourne (AU)

Correspondence Address: BOZICEVIC, FIELD & FRANCIS LLP, 200 MIDDLEFIELD RD, SUITE 200, MENLO PARK, CA, 94025, US

(21) Appl. No.: 10/146,549

(22) Filed: May 13, 2002

Related U.S. Application Data
(60) Provisional application No. 60/290,292, filed on May 11, 2001.

(51) Int. Cl. A61K 38/28; A61K 31/56; A61L 9/04; A61K 38/25

(52) U.S. Cl. 424/45; 514/3; 514/12; 514/177

(57) ABSTRACT

Pegylation or glycosylation of therapeutic proteins to enhance at least one of the solubility, stability and bioavailability thereof, for delivery of an effective amount in an aerosol delivery to the lungs using a minimal number of puffs.

### CROSS-REFERENCE

This application claims the benefit of U.S. Provisional Application No. 60/290,292, filed May 11, 2001, which application is incorporated herein by reference.

### FIELD OF THE INVENTION

The present invention concerns the delivery of proteins by aerosol formulation, and methods and formulations for optimizing delivery of such proteins.

### BACKGROUND OF THE INVENTION

Human growth hormone (e.g., recombinant human growth hormone, rhGH) and other therapeutic proteins such as insulin, testosterone and erythropoeitin are currently given by injection. The administration of such therapeutic proteins by inhalation may require a higher dose delivered because the efficiency of transport from the lung to lymphatics and/or blood circulation may not be as effective as from the injection site. It is also advantageous to give the doses in as small a volume as possible so that the duration of administration is as short as possible for the patient's convenience and to minimize the technical and economic hurdles associated with aerosolization of big volumes of protein formulations. Therefore, it is desirable to achieve as high a concentration of proteins while maintaining their physical and chemical stability as much as possible.

Certain formulation approaches, such as addition of surface-active materials (e.g., Tween 20 and Tween 80, poloxamers, polyethylene glycols) are known to affect the solubility, chemical and physical stability of the therapeutic proteins. For example, U.S. Pat. No. 5,593,844 discloses the inclusion of polysorbates or poloxamers in order to further enhance the stability of a formulation of growth hormone binding protein (GHBP) and growth hormone (GH). Although U.S. Pat. No. 5,593,844 indicates that these formulations may be employed in aerosol devices such as those used in pulmonary dosing, there is no suggestion or enablement of any composition that would be effective for pulmonary dosing. Nor does U.S. Pat. No. 5,593,844 discuss pegylation shows another example of an arrangement for increasing efficiency in a heating element, or any type of covalent bonding with the proteins for pulmonary dosing whatsoever. Thus, U.S. Pat. No. 5,593,844 neither discusses nor solves the problems inherent in aerosol delivery of such formulations, as discussed above, and notes that, most preferably, GHBP and GH are administered subcutaneously by injection, intermittently, every 2 or more days, weekly, biweekly or monthly.

The in vivo half life of certain therapeutic proteins has been increased by conjugating the proteins with polyethylene glycol, a process which is known as pegylation. See e.g., Abuchowski et al., J. Biol. Chem., 252:3578-3586 (1977). PEG is believed to slow renal clearance by providing increased hydrodynamic volume in pegylated proteins. In addition, pegylation has been reported to reduce immunogenicity and toxicity of certain therapeutic proteins.

U.S. Pat. No. 6,136,563 discloses the pegylation of human growth hormone (hGH) variants to increase the half-life thereof in vivo, compared to their non-pegylated counterparts. The pegylated hGH proteins are disclosed as being administered parenterally, and can be administered either locally or systemically. Examples of parenteral administration include subcutaneous, intramuscular, intravenous, intraarterial and intraperitoneal administration. The administration can also be as a single bolus or by slow-release depot formulation. U.S. Pat. No. 6,207,640 also discloses the injection of pegylated growth hormone (GH) using intravenous or subcutaneous means.

Although the addition of surface-active materials (e.g., Tween 20 and Tween 80, poloxamers, polyethylene glycols) or modification by pegylation are known to affect the solubility, chemical and physical stability of the therapeutic proteins, such physical and chemical modifications can also lead to changes in absorption rates (e.g., from changing the association state of the protein, enhancing absorption through effects on the protein conformation or membrane changes, etc.).

Thus, there remains a need for methods and therapeutic formulations for the effective delivery of such formulations in aerosol form, via the airways of a patient. These formulations should be capable of being effectively delivered in one or only a few puffs from an aerosol delivery device, nebulizer or the like.

### SUMMARY OF THE INVENTION

The present invention is directed to aerosols of pegylated or glycosylated protein formulations for delivery to a patient via the lungs, to enhance at least one of the solubility, stability and bioavailability thereof.

The aerosols comprise particles or droplets containing the glycosylated or pegylated protein and having an aerodynamic diameter within the range of about 0.5 to 10 microns, more preferably about 1.0 to 5.0 microns, even more preferably about 1.0 to about 3.5 microns.

The pegylated or glycosylated proteins may be human growth hormone, recombinant human growth hormone, insulin, testosterone, erythropoeitin or other therapeutic protein.

The solubility of the glycosylated or pegylated proteins in an aqueous solution is at least 10% greater than the solubility of a non-glycosylated or non-pegylated form of the same proteins, respectively, more preferably at least 25% greater, still more preferably at least 50% greater.

The molecular weights of the glycosylated or pegylated proteins may be about 5% to about 500% greater than the non-glycosylated or non-pegylated forms of the same proteins, respectively, more preferably about 10% to about 200% greater, still more preferably about 15% to about 100% greater.

The present invention is further directed to pulmonary delivery of proteins that have been pegylated or glycosylated to increase the solubility, stability and/or bioavailability thereof. Example proteins include human growth hormone (e.g., recombinant human growth hormone, rhGH) and other therapeutic proteins such as insulin, testosterone and erythropoeitin. The proteins may be delivered using an inhalation delivery system to deliver particles or droplets containing the pegylated or glycosylated proteins to the peripheral lung.

The pegylated or glycosylated proteins may be manufactured as dry powder with particles predominantly between 0.5 and 10 microns in aerodynamic diameter, preferably between 1 and 5 microns in aerodynamic diameter, more preferably between about 1 and 3.5 microns in aerodynamic diameter.

The pegylation processing of proteins according to the present invention increases the solubility thereof by at least 10%, preferably by 25% and more preferably by 50% or more, as compared to non-pegylated forms of the same proteins, respectively.

Likewise, the glycosylation processing of proteins according to the present invention increases the solubility thereof by at least 10%, preferably by 25% and more preferably by 50% or more, as compared to non-pegylated forms of the same proteins, respectively.

The stability of the proteins in solution or dry state is enhanced by at least 10%, preferably by 25% and more preferably by 50% or more, by pegylation or glycosylation according to the present invention.

The bioavailabilities of the proteins processed by pegylation or glycosylation according to the present invention are improved by at least 10%, preferably by 25% and more preferably by 50% or more.

Pegylation or glycosylation of proteins, according to the present invention, increases the molecular weight of the proteins by at least 5% but not more than 500%, preferably by at least 10% but not more than 200%, most preferably by at least 15% but not more than 100%.

Inhalation delivery systems that may be used to deliver proteins according to the present invention include the AERx® Pulmonary Drug Delivery System, a dry powder inhaler or a nebulizer, for example.

These and other objects, advantages, and features of the invention will become apparent to those persons skilled in the art upon reading the details of the processes and systems as more fully described below.

### DETAILED DESCRIPTION OF PREFERRED EMBODIMENTS

Before the present formulations are described, it is to be understood that this invention is not limited to particular formulations described, as such may, of course, vary. It is also to be understood that the terminology used herein is for the purpose of describing particular embodiments only, and is not intended to be limiting, since the scope of the present invention will be limited only by the claims submitted at such time that this application is converted to a non-provisional application.

Where a range of values is provided, it is understood that each intervening value, to the tenth of the unit of the lower limit unless the context clearly dictates otherwise, between the upper and lower limit of that range and any other stated or intervening value in that stated range is encompassed within the invention. The upper and lower limits of these smaller ranges may independently be included in the smaller ranges is also encompassed within the invention, subject to any specifically excluded limit in the stated range. Where the stated range includes one or both of the limits, ranges excluding either both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

It must be noted that as used herein, the singular forms “a”, “and”, and “the” include plural referents unless the context clearly dictates otherwise. Thus, for example, reference to “a protein” includes a plurality of such proteins and reference to “the hormone” includes reference to one or more hormones and equivalents thereof known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

### DEFINITIONS

The term “pegylation” refers to the binding of various polyethylene glycols (or “PEGs”) to proteins.

The term “glycosylation” refers to the process of adding sugar units such as in the addition of glycan chains to proteins.

The term “GH” is an acronym for growth hormone.

The term “hGH” is an acronym for human growth hormone.

The term “rhGH” is an acronym for recombinant human growth hormone.

The delivery to the lung of therapeutic aerosol formulations, such as those containing proteins, is affected by the particle size of the particles containing the protein. The site of delivery as well as the nature of the formulation affect to what extent the various clearance mechanisms clear the protein from the lung. The various clearance mechanisms include mucociliary clearance, phagocytosis, metabolism, absorption into lymphatics and absorption to the blood stream. Further, if the state of association (e.g., the conformation or the structure around the protein's binding site to its receptor in the body) are affected by the formulation or chemical modification, then different intensity and duration of action of the protein may follow compared to the unformulated, or chemically unmodified protein.

The dosage forms for aerosol delivery of a therapeutic agent to the lungs, such as those provided by Aradigm Corporation of Hayward, California for example, are capable of holding only a small amount of formulation for delivery in a single puff. For example, generally only about 50 μl of a liquid formulation or about 10 mg of fine powder can be provided per individual puff. An additional consideration is the stability of the protein in solution, wherein sufficient stability is needed to prevent the protein from coming out of solution before it is delivered to the target area deep in the lungs.

This invention is therefore about minimizing the volume of the formulation required to achieve a desired therapeutic effect of a protein delivered by pulmonary administration over a given period of time. The minimum volume is obtained with a formulation or a chemical modification in which the solubility (with adequate physical and chemical stability over the proposed shelf-life of the product), respirable fraction, absorption rate, duration of action and potency are maximized while minimizing the competing pathways of drug clearance (metabolism, mucociliary clearance, phagocytosis). An example of such optimization is the preparation of several pegylated derivatives of rhGH, although the present invention is not limited to pegylated rhGH formulations, as pegylated formulations of testosterone, insulin erythropoietin and other therapeutic proteins are contemplated. Procedures for pegylation of therapeutic proteins are described in Bailon and Ehrlich, “Modern-Day Pegylation of Protein Therapeutics”, Hoffman-La Roche Inc., 340 Kingsland Street, Nutley, N.J., which document is incorporated herein, in its entirety, by reference thereto. Additionally, glycosylated proteins, including glycosylated formulations of rhGH, insulin, testosterone, erythropoietin, and other therapeutic proteins are contemplated.

The various pegylated derivatives of rhGH differ in aqueous solubility, stability in vitro, their particle size distribution following the aerosolization of their aqueous solutions, absorption rate and bioavailability following pulmonary administration, binding to the rhGH receptor and the duration of action (which, in turn, is determined by their persistence in the body due to pharmacokinetic and binding properties). The optimum pegylated derivative of rhGH is one that can be delivered in the minimum number of breaths from a system such as AERx (available from Aradigm, Hayward, Calif.) or Respimat (also available from Aradigm), or nebulizer, or other devices that can aerosolize liquid formulations, for the same duration of effective action, provided that such a derivative is sufficiently stable and safe.

An example of an aerosolize that uses an air temperature controlling device for warming air surrounding an aerosolized drug formulation, which may be used for delivering protein formulations according to the present invention, is described in U.S. Pat. No. 6,263,872, which is incorporated herein, in its entirety, by reference thereto.

For proteins where the primary site of action is within the respiratory tract, the optimization therefore actually minimizes the absorption into the lymphatics or the blood stream.

The formulations must be optimized to balance competing factors. For example, an increasing degree of pegylation or glycosylation increases the maximum concentration of protein that can be put into the formulation before the protein aggregates and/or begins to come out of solution. However, at the same time, this increases the hydrophilicity of the particles and may reduce the ability to get the formulation into systemic circulation. Also, an increasing degree of pegylation or glycosylation increases the length of time that the protein molecule stays in the body, but at the same time may lower the biological activity of the molecule. Thus, solubility, duration of action, strength of binding and rate of absorption are all important criteria to be considered in optimizing formulations according to the present invention, with the goal of minimizing the number of puffs required to deliver an effective amount of the formulation by aerosol delivery to the lungs.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

That which is claimed is:

1. A method of providing protein in a form for delivery to a patient via the lungs, to enhance at least one of the solubility, stability and bioavailability thereof, said method comprising the steps of:
providing a protein which has been pegylated; and
aerosolizing the pegylated protein to form particles or droplets having an aerodynamic diameter within the range of about 0.5 to 10 microns.

2. The method of claim 1, wherein the protein is human growth hormone.

3. The method of claim 2, wherein the protein is recombinant human growth hormone.

4. The method of claim 1, wherein the protein is a protein selected from the group consisting of insulin, testosterone and erythropoeitin.

5. The method of claim 1, wherein the aerosolization step is carried out using an AERx system or other inhalation delivery system.

6. The method of claim 1, wherein the aerosolization step is carried out using a dry powder inhaler

7. The method of claim 1, wherein the aerosolization step is carried out using a nebulizer.

8. The method of claim 1, in which the solubility of the pegylated protein in an aqueous solution is at least 10% greater than the solubility of a non-pegylated form of the same protein.

9. The method of claim 8, in which the solubility of the pegylated protein in an aqueous solution is at least 25% greater than the solubility of a non-pegylated form of the same protein.

10. The method of claim 9, in which the solubility of the pegylated protein in an aqueous solution is at least 50% greater than the solubility of a non-pegylated form of the same protein.

11. The method of claim 1, wherein the molecular weight of the pegylated protein is about 5% to about 500% greater than the non-pegylated form of the same protein.

12. The method of claim 11, wherein the molecular weight of the pegylated protein is about 10% to about 200% greater than the non-pegylated form of the same protein.

13. The method of claim 12, wherein the molecular weight of the pegylated protein is about 15% to about 100% greater than the non-pegylated form of the same protein.

14. The method of claim 1, wherein the aerosolized particles or droplets have an aerodynamic diameter within the range of about 1.0 to 5 microns.

15. The method of claim 14, wherein the aerosolized particles or droplets have an aerodynamic diameter within the range of about 1.0 to 3.5 microns.

16. A method of providing protein in a form for delivery to a patient via the lungs, to enhance at least one of the solubility, stability and bioavailability thereof, said method comprising the steps of:
providing a protein which has been glycosylated; and
aerosolizing the glycosylated protein to form particles or droplets having an aerodynamic diameter within the range of about 0.5 to 10 microns.

17. The method of claim 16, wherein the protein is human growth hormone.

18. The method of claim 17, wherein the protein is recombinant human growth hormone.

19. The method of claim 16, wherein the protein is a protein selected from the group consisting of insulin, testosterone and erythropoeitin.

20. The method of claim 16, wherein the aerosolization step is carried out using an AERx system or other inhalation delivery system.

21. The method of claim 16, wherein the aerosolization step is carried out using a dry powder inhaler.

22. The method of claim 16, wherein the aerosolization step is carried out using a nebulizer.

23. The method of claim 16, in which the solubility of the glycosylated protein in an aqueous solution is at least 10% greater than the solubility of a non-glycosylated form of the same protein.

24. The method of claim 23, in which the solubility of the glycosylated protein in an aqueous solution is at least 25% greater than the solubility of a non-glycosylated form of the same protein.

25. The method of claim 24, in which the solubility of the glycosylated protein in an aqueous solution is at least 50% greater than the solubility of a non-glycosylated form of the same protein.

26. The method of claim 16, wherein the molecular weight of the glycosylated protein is about 5% to about 500% greater than the non-glycosylated form of the same protein.

27. The method of claim 26, wherein the molecular weight of the glycosylated protein is about 10% to about 200% greater than the non-glycosylated form of the same protein.

28. The method of claim 27, wherein the molecular weight of the glycosylated protein is abou t 15% to about 100% greater than the non-glycosylated form of the same protein.

29. The method of claim 16, wherein the aerosolized particles or droplets have an aerodynamic diameter within the range of about 1.0 to 5 microns.

30. The method of claim 29, wherein the aerosolized particles or droplets have an aerodynamic diameter within the range of about 1.0 to 3.5 microns.

31. An aerosol of a pegylated protein formulation for delivery to a patient via the lungs, to enhance at least one of the solubility, stability and bioavailability thereof, said formulation comprising:
particles or droplets containing the pegylated protein and having an aerodynamic diameter within the range of about 0.5 to 10 microns.

32. The aerosol of claim 31, wherein the protein is human growth hormone.

33. The aerosol of claim 32, wherein the protein is recombinant human growth hormone.

34. The aerosol of claim 31, wherein the protein is a protein selected from the group consisting of insulin, testosterone and erythropoeitin.

35. The aerosol of claim 1, in which the solubility of the pegylated protein in an aqueous solution is at least 10% greater than the solubility of a non-pegylated form of the same protein.

36. The aerosol of claim 35, in which the solubility of the pegylated protein in an aqueous solution is at least 25% greater than the solubility of a non-pegylated form of the same protein.

37. The aerosol of claim 36, in which the solubility of the pegylated protein in an aqueous solution is at least 50% greater than the solubility of a non-pegylated form of the same protein.

38. The aerosol of claim 31, wherein the molecular weight of the pegylated protein is about 5% to about 500% greater than the non-pegylated form of the same protein.

39. The aerosol of claim 38, wherein the molecular weight of the pegylated protein is about 10% to about 200% greater than the non-pegylated form of the same protein.

40. The aerosol of claim 39, wherein the molecular weight of the pegylated protein is about 15% to about 100% greater than the non-pegylated form of the same protein.

41. The aerosol of claim 31, wherein the particles or droplets have an aerodynamic diameter within the range of about 1.0 to 5 microns.

42. The aerosol of claim 41, wherein the particles or droplets have an aerodynamic diameter within the range of about 1.0 to 3.5 microns.

43. An aerosol of a glycosylated protein formulation for delivery to a patient via the lungs, to enhance at least one of the solubility, stability and bioavailability thereof, said formulation comprising:
particles or droplets containing the glycosylated protein and having an aerodynamic diameter within the range of about 0.5 to 10 microns.

44. The aerosol of claim 43, wherein the protein is human growth hormone.

45. The aerosol of claim 44, wherein the protein is recombinant human growth hormone.

46. The aerosol of claim 43, wherein the protein is a protein selected from the group consisting of insulin, testosterone and erythropoeitin.

47. The aerosol of claim 43, in which the solubility of the glycosylated protein in an aqueous solution is at least 10% greater than the solubility of a non-glycosylated form of the same protein.

48. The aerosol of claim 47, in which the solubility of the glycosylated protein in an aqueous solution is at least 25% greater than the solubility of a non-glycosylated form of the same protein.

49. The aerosol of claim 48, in which the solubility of the glycosylated protein in an aqueous solution is at least 50% greater than the solubility of a non-glycosylated form of the same protein.

50. The aerosol of claim 43, wherein the molecular weight of the glycosylated protein is about 5% to about 500% greater than the non-glycosylated form of the same protein.

51. The aerosol of claim 50, wherein the molecular weight of the glycosylated protein is about 10% to about 200% greater than the non-glycosylated form of the same protein.

52. The aerosol of claim 51, wherein the molecular weight of the glycosylated protein is about 15% to about 100% greater than the non-glycosylated form of the same protein.

53. The aerosol of claim 43, wherein the particles or droplets have an aerodynamic diameter within the range of about 1.0 to 5 microns.

54. The aerosol of claim 53, wherein the particles or droplets have an aerodynamic diameter within the range of about 1.0 to 3.5 microns.
